(19) United States
(12) Patent Application Publication
(10) Pub. No.: US 2009/0221954 A1
(43) Pub. Date: Sep. 3, 2009

(54) IONTOPHORESIS DEVICE

(76) Inventors: Mitsugu Yamauchi, Tokyo (JP); Rie Yamamoto, Tokyo (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 12/393,890

(22) Filed: Feb. 26, 2009

Related U.S. Application Data
(63) Continuation-in-part of application No. PCT/JP2007/062027, filed on Jun. 14, 2007.

(30) Foreign Application Priority Data
Aug. 30, 2006 (JP) 2006-233662

(51) Int. Cl. A61N 1/30

(52) U.S. Cl. 604/20

(57) ABSTRACT

An iontophoresis device (includes a working electrode assembly and a non-working electrode assembly, each having respective organism contact surfaces. A release liner may be peeled off when it is pulled in one direction along the contact surfaces. A base film may be coupled to a front surface of the release liner and may be sized to cover the front regions of the contact surfaces. A paste layer is attached to the front surface of the base film. The paste layer is peeled from the skin by pulling the base film via the release liner. The paste layer retains a part of the stratum corneum or sebum when removed from the skin.

### CROSS REFERENCE TO RELATED APPLICATION

This is a continuation-in-part application of PCT/JP2007/062027 filed on Jun. 14, 2007, the contents of which are incorporated herein by reference in its entirety.

### BACKGROUND

1. Technical Field

The present application relates to iontophoresis devices for administering a drug ion to an organism by applying a voltage.

2. Description of the Related Art

PCT patent application publication WO 0303742 describes an iontophoresis device as including a DC power supply, a working side electrode assembly, and a non-working side electrode assembly, for administering a drug solution to skin or mucosa from an ion exchange membrane of the working side electrode assembly.

The working side electrode assembly is described as being formed by stacking a first ion exchange membrane constituting an organism contact surface to contact the skin or mucosa, a drug solution holding portion, a second ion exchange membrane, a first electrolyte solution holding portion, and a working side electrode assembly in the stated order.

The non-working side electrode assembly is described as being formed by stacking a fourth ion exchange membrane constituting an organism contact surface to contact the skin or mucosa, a third electrolyte solution holding portion, a third ion exchange membrane, a second electrolyte solution holding portion, and a non-working side electrode in the stated order.

It is naturally required for the organism contact portions of the working side electrode assembly and the non-working side electrode assembly to be in contact with the skin or mucosa in order to effectively administer the drug solution. However, on the working side electrode assembly side, administration efficiency of the drug solution ion is largely varied depending on dryness of the skin, quantity of sebum, thickness of stratum corneum, or the like.

To address such, it is conceivable to bring the working side electrode assembly and the non-working side electrode assembly into contact with the skin or mucosa after removing the sebum and the stratum corneum from the skin as much as possible.

No difficulty occurs if the sebum and skin removing operation is conducted by a nurse or the like at a medical institution. However, the above-mentioned removing operation is difficult if the iontophoresis device is used by a user by himself at his home, or the like, and will not likely be conducted.

### BRIEF SUMMARY

In view of the above-mentioned problem, applicants described herein various embodiments of an iontophoresis device which is constructed so that the iontophoresis device may be used after removing the sebum and the stratum corneum partially or fully at a region for administering the drug solution ion on the skin.

An iontophoresis device may be summarized as including: a power source; and a working side electrode assembly and a non-working side electrode assembly connected to the power source, for administering a drug ion held in the working side electrode assembly to an organism from an organism contact surface at a tip of the working side electrode assembly through voltage applied from the power source, in which: the iontophoresis device further includes a release liner which is attached so as to cover the organism contact surface and is peelable by pulling the release liner in one direction along the organism contact surface; and on a front surface side of the release liner, in the organism contact surface, a base film, which is connected to the release liner at a vicinity of an end portion opposite to the pulling direction side and which covers at least a front surface region of the organism contact surface, and a paste layer attached to the front surface of the base film, the paste layer having an adhesive force which is peelable with respect to the skin by pulling the base film toward the pulling direction. Thus, the above-mentioned problem may be addressed by mounting the device after removing even a little amount of the stratum corneum and the sebum of the skin prior to use of the device.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is an exploded perspective view illustrating an iontophoresis device according to Embodiment 1.

FIG. 2 is a sectional view of the iontophoresis device of FIG. 1.

FIG. 3 is a sectional view schematically illustrating a process of exfoliating a base film in Embodiment 1.

FIG. 4 is a sectional view schematically illustrating an iontophoresis device according to Embodiment 2.

FIG. 5 is a sectional view schematically illustrating an iontophoresis device according to Embodiment 3.

FIG. 6 is a sectional view schematically illustrating an iontophoresis device according to Embodiment 4.

FIG. 7 is a sectional view schematically illustrating an exfoliating process for a paste layer in Embodiment 4.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with drug delivery devices, transdermal devices and iontophoresis devices have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

Hereinafter, description is made of Embodiments 1 to 4.

### Embodiment 1

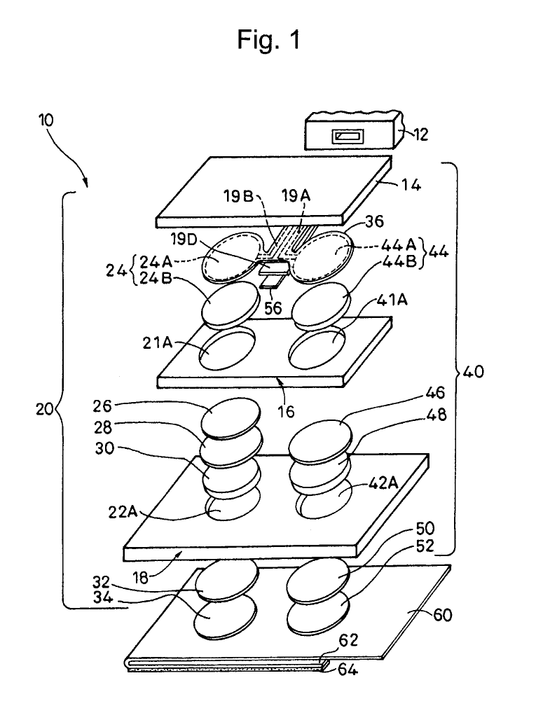

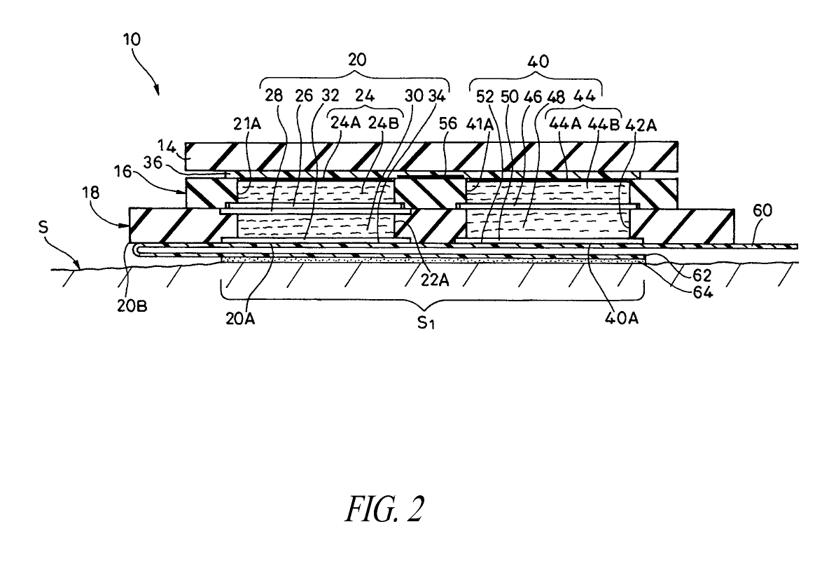

An iontophoresis device 10 according to an Embodiment 1 will be described in detail with reference to FIGS. 1 to 2.

The iontophoresis device 10 comprising a DC electric power source 12, a working electrode assembly 20 connected to one of an anode or cathode of the DC electric power source 12, and a non-working electrode assembly 40 connected to the other of the anode or cathode. The device administers a drug ion held by the working electrode assembly 20 to an organism with a voltage from the DC electric power source 12 through an organism contact surface 20A which being a tip surface of the working electrode assembly 20.

In the iontophoresis device 10, each of the working electrode assembly 20 and the non-working electrode assembly 40 is constructed by interposing constituent members such as a drug solution holding portion between a base end support 14 and an intermediate support 16, which are overlapped and are each formed of a resin sheet, for example, polyurethane foam sheet, or by receiving the constituent members in through-holes formed in the intermediate support 16 and a tip support portion 18. The base end support 14 and the intermediate support 16 are formed into the same size, and the tip support 18 is formed so as to be larger than the supports 14 and 16.

With reference to FIG. 2, the base end support 14, the intermediate support 16, and the tip support 18 each have respective pressure-sensitive adhesive layers 14A, 16A, and 18A, respectively, at the lower surfaces thereof so as to be stuck to one another. At the same time, the pressure-sensitive adhesive layer 18A of the tip support 18 may be selectively stuck to the skin or mucosa in use.

Here, the intermediate support 16 is a single sheet-like member which forms a part of the working electrode assembly 20 and a part of the non-working electrode assembly 40.

Similarly, the tip support 18 is a single sheet-like member which forms a part of the working electrode assembly 20 and a part of the non-working electrode assembly 40.

The organism contact surface 20A of the working electrode assembly 20 and the organism contact surface 40A which being a tip surface of the non-working electrode assembly 40 are arranged flush with each other and with a gap there between. Further, there is provided a release liner 60 so as to cover the organism contact surfaces 20A and 40A which, as previously noted, are flush with each other.

The release liner 60 is peelable by pulling in one direction along the organism contact surfaces 20A and 40A, for example, in a right direction in FIG. 1.

Further, on a front surface side of the release liner 60, in the organism contact surfaces 20A are provided, a base film 62, which is connected to the release liner 60 at a vicinity of an end portion 20B opposite to the pulling direction side and has a size capable of covering at least a front surface region of the organism contact surfaces 20A and 40A, and a paste layer 64 attached to the front surface of the base film 62.

The paste layer 64 has an adhesive force which is peelable with respect to the skin S by pulling the base film 62 toward the pulling direction, and is constructed so that a part of the stratum corneum and the sebum of the surface of the skin S may be simultaneously peeled off when the paste layer 64 is peeled off from the skin S.

The base film 62 is formed by bending the release liner 60 into a U shape at the vicinity of the end portion 20B and by extending the release liner. Here, the paste layer 64 is preferably made of a material which adheres to the skin S but is hard to adhere to the organism contact surfaces 20A and 40A.

The structures of the working electrode assembly 20 and the non-working electrode assembly 40 are further described in detail.

The working electrode assembly 20 is formed by stacking a working electrode 24 connected to an anode and a cathode in a DC electric power source 12, which is the same polarity with a drug ion, a separator 26 arranged on a front surface of the working electrode 24, a second ion selective membrane 28 arranged on a front surface of the separator 26, selectively passing an ion opposite in polarity to the drug ion, a drug solution holding portion 30 arranged on a front surface of the second ion selective membrane 28, for holding a drug which is different from the drug ion, a first ion selective membrane 32 arranged on a front surface of the drug solution holding portion 30, selectively passing an ion which is the same polarity as the drug ion, and a working organism contact portion 34 which is formed by applying a viscous liquid containing a drug which is the same with the drug on a front surface of the first ion selective membrane 32 in the stated order. A front end surface of the working organism contact portion 34 is constituted as the working organism contact surface 20A which is brought into contact with the skin or mucosa.

The working electrode 24 comprises: a working collector 24A connected to the DC electric power source 12 and including a carbon-printed electrode formed by printing on the front surface of a resin sheet 36; and a working polarizable electrode 24B electrically connected to the front surface of the working collector 24A.

It should be noted that the phrase “electrically connected” comprehends not only the case where the electrode is directly connected to the front surface but also the case where the electrode is connected to the front surface through an electric conductor such as a conductive adhesive (the same holds true for the following).

The intermediate support 16 is formed of a resin material having a thickness substantially equal to that of the working polarizable electrode 24B, and has a working intermediate through-hole 21A of substantially the same shape as that of an external shape in a plane shape of the working polarizable electrode 24B. The working polarizable electrode 24B is received in the working intermediate through-hole 21A.

In addition, the tip support 18 is formed of a resin material having a thickness substantially equal to that of the drug solution holding portion 30, and has a working tip through-hole 22A of substantially the same shape as that of an external shape in the plane shape of the working polarizable electrode 24B. The drug solution holding portion 30 is received in the working tip through-hole 22A.

The non-working electrode assembly 40 is formed by stacking, from the base end support 14 in the following order, a non-working electrode 44 connected to an anode or a cathode in a DC electric power source 12, which is of an opposite polarity to that of the drug ion, a separator 46 arranged on a front surface of the non-working electrode 44, an electrolyte solution holding portion 48 for holding an electrolyte solution, a third ion selective membrane 50 selectively passing an ion opposite in polarity to a polarity of the drug ion, and a non-working organism contact portion 52 which is formed by applying a viscous liquid containing an electrolyte solution which is the same type as the electrolyte solution which is held in the electrolyte solution holding portion 48. A front end surface of the non-working organism contact portion 52 is constituted as the non-working organism contact surface 40A.

The non-working electrode 44 comprises: a non-working collector 44A which is formed of a material containing carbon and printed in a film shape on the front surface of the resin sheet 36 so as to be distinct from the working collector 24A of the working electrode 24; and a non-working polarizable electrode 44B provided so as to contact the non-working collector 44A.

The non-working polarizable electrode 44B has a thickness equal to that of the intermediate support 16, and is received in a non-working intermediate through-hole 41A formed in the intermediate support 16. In addition, the electrolyte solution holding portion 48 has a thickness equal to that of the tip support 18, and is received in a non-working tip through-hole 42A formed in the tip support 18.

In Embodiment 1, each of the through-holes 22A, 21A, 41A, and 42A is of a circular shape, and, furthermore, each of the working electrode 24, the separator 26, the second ion selective membrane 28, the drug solution holding portion 30, the first ion selective membrane 32, and the working organism contact portion 34 is of a circular membrane shape or a sheet shape.

Similarly, each of the non-working electrode 44, the separator 46, the electrolyte solution holding portion 48, the third ion selective membrane 50, and the non-working organism contact portion 52 is of a circular membrane shape or a sheet shape.

As illustrated in FIG. 1 by a broken line, in the resin sheet 36, a working wire 19A and a non-working wire 19B which are formed of a material containing carbon and printed continuously in a film shape are connected to the working collector 24A in the working electrode 24 and the non-working collector 44A in the non-working electrode 44, respectively

In Embodiment 1, as shown in each of FIGS. 1 and 4, the respective circular members are overlapped in each of the working electrode assembly 20 and the non-working electrode assembly 40 in the thickness direction of each assembly, and are integrated to constitute the iontophoresis device 10.

Here, the intermediate support 16 is interposed between other members from above and below the support in a state where the working polarizable electrode 24B and the non-working polarizable electrode 44B are received in the working intermediate through-hole 21A and non-working intermediate through-hole 41A of the intermediate support 16, respectively. Similarly, the tip support 18 is interposed between other members from above and below the support in a state where the drug solution holding portion 30 and the electrolyte solution holding portion 48 are stored in the working tip through-hole 22A and non-working tip through-hole 42A of the tip support 18, respectively. Further, a member between the base end support 14 and the intermediate support 16 is positioned and fixed by being interposed between them, and a member between the intermediate support 16 and the tip support 18 is positioned and fixed by being interposed between them.

Each of outer diameters of the working collector 24A and the non-working collector 44A in the working electrode 24 and the non-working electrode 44 is slightly larger than each of diameters of the working intermediate through-hole 21A and non-working intermediate through-hole 41A. Outer peripheral portions thereof are arranged so as to be sandwiched between the base end support 14 and the intermediate support 16.

Reference numeral 56 of FIG. 1 and FIG. 2 represents an adhesive. The adhesive 56 is placed across an intermediate portion between the working and non-working collectors 24A and 44A in the insulating film 19D to bind the insulating film 19D and the intermediate support 16 so that the area between the insulating film 19D and the intermediate support 16 is partitioned into a working side and a non-working side.

In addition, reference numeral 60 of FIG. 1 represents a release liner attached to the front surface of the tip support 18 in a peelable fashion so as to cover the working organism contact portion 34 and the non-working organism contact portion 52.

Next, materials, components, and the like for the above respective constituent elements are described.

In Embodiment 1, the drug solution holding portion 30 is constructed by impregnating a polypropylene (PP) non-woven fabric with a viscous liquid containing the drug. In addition, the drug solution holding portion 30 is impregnated with a drug, the drug effect component of which dissociates into a positive or negative ion by, for example, dissolution in a solvent such as water (drug which dissociates into a drug ion (a precursor for the drug is also permitted)). A drug, the drug effect component of which dissociates into a positive ion is, for example, lidocaine hydrochloride as a narcotic or morphine hydrochloride as a narcotic. A drug, the drug effect component of which dissociates into a negative ion is, for example, ascorbic acid which being a vitamin agent.

In addition to the foregoing, a hormone, DNA, RNA, a protein, an amino acid, and minerals are also included in the category described above.

The separator 46 in the non-working electrode assembly 40 is obtained by impregnating a PP non-woven fabric with a viscous liquid containing the electrolyte solution (details about the electrolyte solution are described later). Further, the electrolyte solution holding portion 48 is obtained by impregnating a PP non-woven fabric with a viscous liquid containing the same electrolyte solution.

The electrolyte solution to be used in each of the separator 46 and the electrolyte solution holding portion 48 is mainly formed of an electrolyte, and electrolytes each of which is oxidized or reduced more readily than the electrolytic reaction of water (oxidation at an anode and reduction at a cathode) such as: pharmaceuticals such as ascorbic acid (vitamin C), and sodium ascorbate; and organic acids such as lactic acid, oxalic acid, malic acid, succinic acid, and fumaric acid and/or salts of the acids are each particularly preferably used as the electrolyte. As a result, the generation of an oxygen gas or hydrogen gas may be suppressed. In addition, a fluctuation in pH of the electrolyte solution during energization may be suppressed by blending such a combination of multiple kinds of electrolytes as to provide a buffer electrolyte solution when being dissolved in a solvent.

Such viscous liquid containing the drug or electrolyte solution described above may be produced by, for example, mixing water (ion exchange water) with 2 mass % or more of a tacky material such as hydroxypropylcellulose (HPC) (for example, H-Type manufactured by NIPPON SODA CO., LTD.) or a Metolose as a water-soluble polymer obtained by chemically treating a water-insoluble cellulose (for example, 90SH-10000SR manufactured by Shin-Etsu Chemical Co., Ltd.).

The working polarizable electrode 24B in the working electrode 24 and the non-working polarizable electrode 44B in the non-working electrode 44 are either constructed of as a main component or a conductive base, which is formed of active carbon, preferably, a carbon fiber or carbon fiber paper. If only an active carbon fiber is used as the working and non-working polarizable electrodes 24B and 44B, it is preferred to form a layer by combining cloth and felt formed of the active carbon fiber. Beside, for the conductive base, for example, layers in which the active carbon is dispersed within a binder polymer may be laminated. Active carbon having a specific area of 10 m²/g or more may be used.

The working polarizable electrode 24B is impregnated with a viscous liquid containing a drug identical to the drug held by the drug solution holding portion 30. In addition, the non-working polarizable electrode 44B is impregnated with a viscous liquid containing an electrolyte solution identical to the electrolyte solution held by the separator 46.

Each of the working collector 24A in the working electrode 24 and the non-working collector 44A in the non-working electrode 44 may be a printed electrode obtained by printing a polyethylene terephthalate (PET) material mixed with carbon and an adhesive.

It should be noted that, any electrically conductive material may be used for the working collector 24 and the non-working collector 44A, for example, a metal such as gold, platinum, silver, copper, or zinc other than carbon may be used. Alternatively, a conductive material such as carbon or gold itself may be used as a collector without reliance on printing. In addition, as a material for the working wire 19A and the non-working wire 19B, the same may be applied.

The separator 26 is obtained by impregnating a PP non-woven fabric with a viscous liquid containing a drug identical to the drug held by the drug solution holding portion 30, and is interposed between the working polarizable electrode 24B and the second ion selective membrane 28 to prevent physical contact between them.

The first ion selective membrane 32 is constructed by incorporating an ion-exchange resin into which an ion-exchange group is introduced so that an ion having the same polarity as the drug ion selectively substantially passes through the resin while ions of the opposite polarity are substantially blocked. Specifically, the first ion selective membrane 32 contains a cation exchange resin when the drug solution of the drug solution holding portion 30 dissociates into a cation, and contains an anion exchange resin when the drug solution dissociates into an anion.

The second ion selective membrane 28 is constructed by incorporating an ion-exchange resin into which an ion-exchange group is introduced so that an ion of opposite polarity as the drug ion selectively substantially passes through the resin while ions of the same polarity as the drug are substantially blocked. Specifically, the second ion selective membrane 28 contains an anion exchange resin when the drug solution of the drug solution holding portion 30 dissociates into a cation, and contains the cation exchange resin when the drug solution dissociates into the anion.

As in the case of the second ion selective membrane 28, the third ion selective membrane 50 is constructed by incorporating an ion exchange resin into which an ion exchange group is introduced so that an ion opposite in polarity to the drug ion selectively passes through the resin. That is, when the drug solution of the drug solution holding portion 30 dissociates into a cation, the third ion selective membrane 50 contains an anion exchange resin, and when the drug solution dissociates into an anion, the membrane contains a cation exchange resin.

As the above cation exchange resin, there may be used, without any limitation, an ion exchange resin obtained by introducing a cation exchange group (exchange group the counter ion of which is a cation) such as a sulfonic group, a carboxylic group, or a phosphonic group into a polymer having a three-dimensional network structure such as a hydrocarbon-based resin such as a polystyrene resin or an acrylic acid-based resin, or a fluorine-based resin having a perfluorocarbon skeleton.

In addition, as the above anion exchange resin, there may be used, without any limitation, an ion exchange resin obtained by introducing a cation exchange group (exchange group, the counter ion of which is an anion) such as a primary, secondary, or tertiary amino group, a quaternary ammonium group, a pyridyl group, an imidazole group, a quaternary pyridium group, or a quaternary imidazolium group into the same polymer having a three-dimensional network structure as that in the case of the cation exchange resin.

The working organism contact portion 34 is constructed by applying a viscous liquid, identical to a liquid with which the drug solution holding portion 30 is impregnated, to the front surface of the first ion selective membrane 32. In addition, the non-working organism contact portion 52 is constructed by applying a viscous liquid containing an electrolyte solution identical to that used in the electrolyte solution holding portion 48 to the front surface of the third ion selective membrane 50. It should be noted that each of those viscous liquids is desirably applied in an amount as small as 7 μL in order that the spread of each of the applied viscous liquids due to the attachment of the release liner 60 on each of the viscous liquids may be suppressed.

At the time of the assembly of the iontophoresis device 10, in a state shown in FIG. 1, the respective constituent members are arranged like a laminate or stored in through-holes, and the base end support 14, the intermediate support 16, and the tip support 18 are sequentially overlapped on the release liner 60, and are fixed by being stuck to one another with the pressure-sensitive adhesive layers 16A and 14A. Thus, the assembly is completed.

A button cell or a thin cell disclosed in, for example, Japanese Patent Application Laid-Open No. Hei 11-067236, US Patent Publication No. 2004/0185667 A1, or U.S. Pat. No. 6,855,441 may be used as the DC electric power source 12, although other types of power sources may be employed in this embodiment.

When the above-mentioned iontophoresis device 10 is used, as illustrated in FIG. 2, the paste layer 64 of the base film 62 is adhered to a contact region Si, which is a portion of the skin S, with which the working organism contact surface 20A and the non-working organism contact surface 40A (hereinafter, referred to as organism contact surfaces 20A and 40A) are brought into contact.

Next, the release liner 60 is pulled (e.g., toward the right as viewed in FIG. 2) substantially parallel to the organism contact surfaces 20A and 40A. In this case, for example, a user grasps the whole iontophoresis device 10, to thereby fix its position with respect to the skin S.

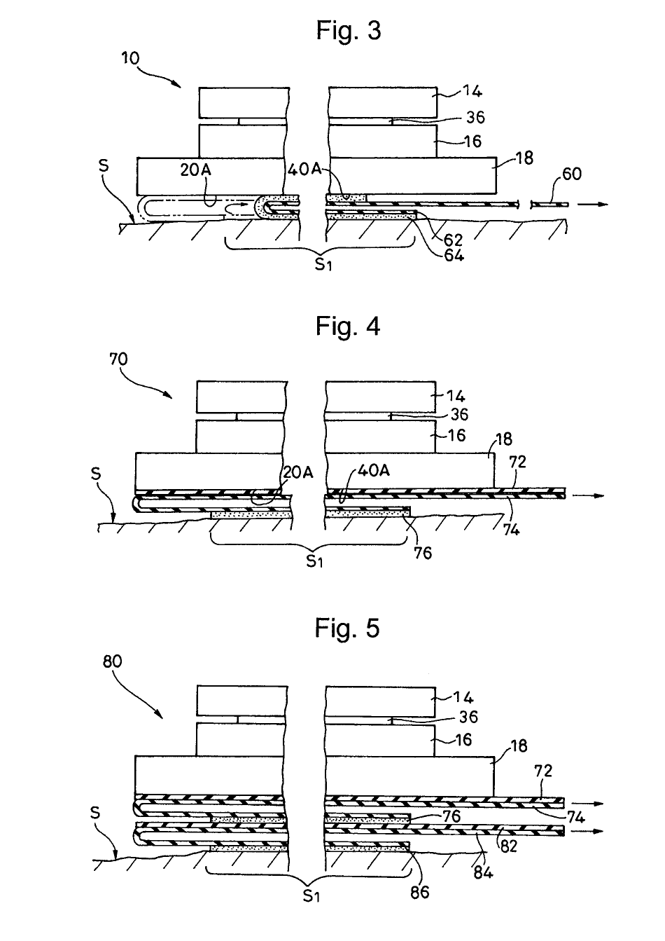

With this, the release liner 60 moves along the organism contact surfaces 20A and 40A, and the base film 62 is also pulled through the movement of the release liner 60, with the result that the paste layer 64 is, as illustrated in FIG. 3, peeled from the skin S sequentially from the left side. A part of the stratum corneum or sebum of the skin S is adhered to the paste layer 64. This means that the stratum corneum or sebum is peeled from the contact region Si of the skin S.

The paste layer 64 peeled from the skin S moves along the organism contact surfaces 20A and 40A (toward the right as viewed in the figure), and when the ends (right ends in the figure) of the base film 62 and the paste layer 64 pass through the organism contact surfaces 20A and 40A, the peeling of the release liner 60 and the base film 62 are finished.

In this state, the iontophoresis device 10 is mounted so that the organism contact surfaces 20A and 40A are brought into contact with the skin S.

In the contact region Si with which those organism contact surfaces 20A and 40A are brought into contact, the part of the stratum corneum or sebum has been peeled as described above, and hence impedance of the skin is small and the drug ion may be administered efficiently.

### Embodiment 2

An iontophoresis device 70 according to Embodiment 2 will be described with reference to FIG. 4.

The iontophoresis device 70 comprises the release liner 60, a release liner 72 and a base film 74, each having a different structure from the base film 62. The other structure is the same as the structure of the iontophoresis device 10 according to Embodiment 1, and hence the detailed description of such is not repeated.

The release liner 72 according to Embodiment 2 is, as illustrated in FIG. 4, formed as a separate member from the base film 74, and at least a portion of the base film 74 is integrally formed on a front surface of the release liner 72. In addition, the base film, 74 is bent into a U-shape at the vicinity of an end portion 20B to be extended, and the paste layer 76 is formed on the extended portion.

In Embodiment 2, when the release liner 72 and the base film 74 which is integrally formed therewith are pulled (toward the right as viewed in FIG. 4), while supporting the iontophoresis device 70, the paste layer 76 is sequentially peeled from the contact region Si of the skin S from a left end (as viewed in FIG. 4). At this time, a part of the stratum corneum or sebum of the skin S may be peeled off.

In contrast to Embodiment 1, in Embodiment 2 the peeled paste layer 76 is free from contact with the organism contact surfaces 20A and 40A, and hence the peeling of the release liner 72 from the organism contact surfaces 20A and 40A advantageously may be performed more smoothly, and a range of material suitable for the paste layer 76 advantageously becomes wider.

### Embodiment 3

An iontophoresis device 80 according to Embodiment 3 will be discussed with reference to FIG. 5.

The iontophoresis device 80 is constructed by adding a second release liner 82, a second base film 84, and a second paste layer 86 with respect to the iontophoresis device 70 of Embodiment 2 (FIG. 4), and the other structures are the same as the iontophoresis device 70 according to Embodiment 2. Accordingly, the detailed description thereof is not repeated.

The second release liner 82 in Embodiment 3 is provided on a lower side (with respect to the orientation illustrated in FIG. 5) of the paste layer 76 which is attached to the base film 74. The second base film 84 is integrally formed with respect to the second release liner 82 on the front surface of the second release liner 82, and the second base film 84 is also bent into a U-shape at the vicinity of the end portion 20B to be extended.

Specifically, the second release liner 82, the second base film 84, and the second paste layer 86 each have the same structure as the release liner 72, the base film 74, and the paste layer 76, respectively, of Embodiment 2, are additions to the stack structure of Embodiment 2.

To use iontophoresis device 80 of Embodiment 3, first, the second release liner 82 and the second base film 84 on the skin S side are pulled toward the right (as viewed in FIG. 5), thereby peeling off the second paste layer 86 from the skin S, and the second release liner 82 is peeled off from the paste layer 76 above the second release liner 82.

Next, after adhering the paste layer 76 of the base film 74 onto the skin S, the release liner 72 and the base film 74 are pulled toward the right (as viewed in FIG. 5), whereby the paste layer 76 peels off a part of the stratum corneum or sebum from the skin S as described above.

Like this, the part of the stratum corneum or sebum of the skin S are peeled off two times by the paste layers 86 and 76, respectively, and hence, compared to a case where the peeling is conducted one time, the impedance of the skin may be made even smaller.

In Embodiment 3 (FIG. 5), the part of the stratum corneum or the like is peeled off two times from the skin S, respectively, by two layers of the paste layers 86 and 76. However, embodiments of the present invention are not limited thereto, and a combination of the release liner, base film, and the paste layer may be provided in three layers.

### Embodiment 4

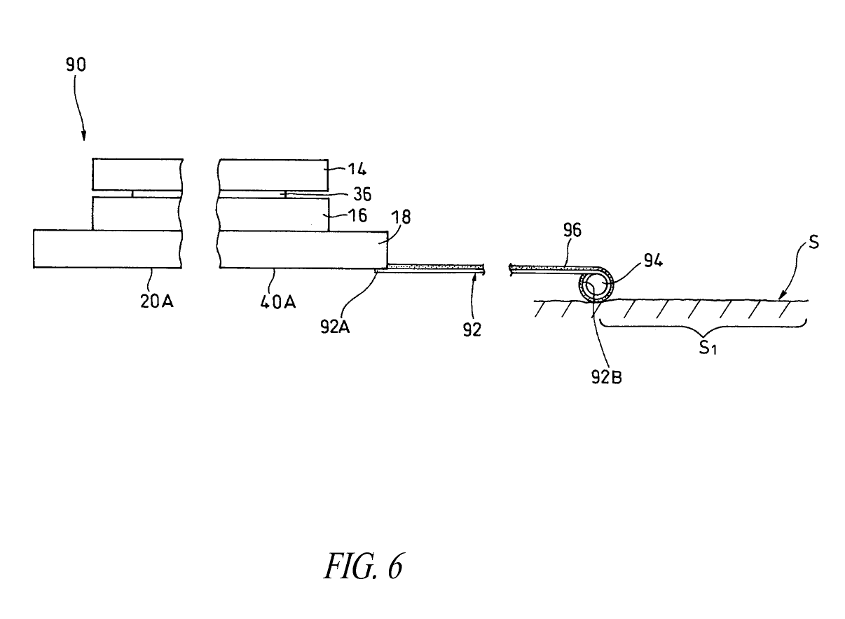

An iontophoresis device 90 according to Embodiment 4 will be discussed with reference to FIG. 6.

The iontophoresis device 90 has a substantially same shape and size (a total of the organism contact surfaces 20A and 40A), and includes: a release liner 92 having one side end 92A which is attached at a right end portion of the non-working organism contact surfaces 40A (as viewed in FIG. 6); and a winding member 94 attached to another side end 92B, which is opposite to the one side end 92A of the release liner 92, capable of winding the release liner 92, while rotating, from the another side end 92B toward the one side end 92A.

The release liner 92 has a paste layer 96 formed on a surface which is opposite to a surface which is brought into contact with the winding member 94, and constitutes a paste-attached liner. The paste layer 96 is constructed so as to have an adhesive force which is peelable with respect to the skin when the paste layer-attached release liner is rolled up.

In the iontophoresis device 90 according to Embodiment 4, the paste layer 96 outside the winding member 94 is brought into contact with the contact region Si of the skin S, with which the organism contact surfaces 20A and 40A are to be brought into contact, from a left side (as viewed in FIG. 6), and then the winding member 94 is rotated, for example, toward the right (as viewed in FIG. 6).

If the winding member 94 is rotated toward the right (as viewed in FIG. 6), the outside paste layer 96 is brought into contact with the contact region Si, and further, when the paste layer 96 is taken up by the winding member 94, a part of the stratum corneum or sebum of the contact region Si is peeled off through the adhesive force of the paste layer 96.

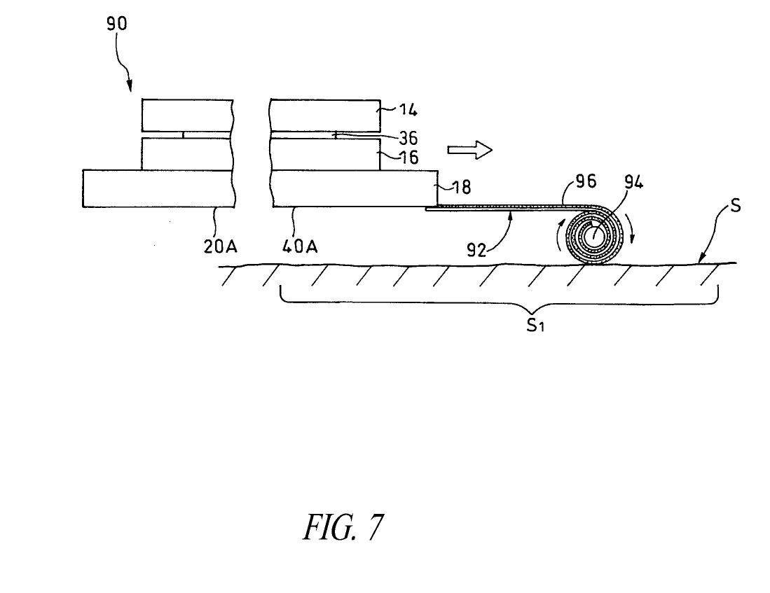

Through a way as described above, when the winding member 94 is caused to advance while taking up the release liner 92 along the contact region Si, as illustrated in FIG. 7, the part of the stratum corneum or sebum in an entire area of the contact region Si is peeled off by the paste layer 96.

At a time of finish of the peeling, the organism contact surfaces 20A and 40A are positioned on an upper side of the contact region Si. If electrification is conducted after removing the release liner 92 or as it is, without removing, and bringing the organism contact surfaces 20A and 40A into contact with the contact region Si, the drug ion may efficiently be administered.

It should be noted that, in each of the iontophoresis devices 10, 70, 80, and 90 according to the above-mentioned embodiments, each of the paste layers 64, 76, 86, and 96 peels off a part of the stratum corneum, or the like from the contact region Si of the skin S in an area where the both the organism contact surfaces 20A and 40A are brought into contact with the paste layers 64, 76, 86, and 96, but the embodiments of the present invention are not limited to this. The part of the stratum corneum, or the like may only be peeled off from the skin S in at least an area with which the working contact surface 20A of the working electrode assembly is brought into contact.

Further, the structure of the iontophoresis device main body is not limited to the structures according to Embodiments 1 to 4, but may be applied, in the working electrode assembly, to a case of including at least a working electrode and a drug solution holding portion.

Further, the structure of the iontophoresis device main body may be applied to a case where, without using the working organism contact portion 34 or non-working organism contact portion 52, the ion selective film constitutes the organism contact surface.

A transdermal drug delivery devices allows a user or patient themselves to selectively remove a base film which is adhered with respect to the skin or mucous membrane through an adhesive or paste layer, by using a release liner covering an organism contact surface at least at a tip of a working electrode assembly and a non-working electrode assembly, and thereby peel off part of a stratum corneum or sebum from a drug solution ion administering area by causing the stratum corneum or sebum to be attached to the adhesive or paste layer. As a result, the device is easy to use.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The teachings provided herein of the various embodiments can be applied to other drug delivery devices, not necessarily the exemplary iontophoresis delivery device generally described above.

The various embodiments described above can be combined to provide further embodiments. Aspects of the embodiments can be modified, if necessary, to employ systems, circuits and concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An iontophoresis device, comprising: a power source; and a working side electrode assembly and a non-working side electrode assembly connected to the power source, for administering a drug ion held in the working side electrode assembly to an organism from an organism contact surface at a tip of the working side electrode assembly through voltage applied from the power source, wherein:
the iontophoresis device further comprises a release liner which is attached so as to cover the organism contact surface and is peelable by pulling the release liner in one direction along the organism contact surface; and
on a front surface side of the release liner, in the organism contact surface, a base film, which is connected to the release liner at a vicinity of an end portion opposite to the pulling direction side and covers at least a front surface region of the organism contact surface, and a paste layer attached to the front surface of the base film are provided, the paste layer having an adhesive force which is peelable with respect to the skin by pulling the base film toward the pulling direction.

2. The iontophoresis device according to claim 1, wherein the base film is formed by bending the release liner into a U shape at the vicinity of the end portion and by extending the release liner.

3. The iontophoresis device according to claim 2, further comprising:
a second release liner which is attached to a front surface of the paste layer so as to cover the paste layer, and is peelable by pulling the second release liner along the paste layer; and
a second base film which is provided on a front surface side of the second release liner, in the paste layer, which is connected to the second release liner at least at a vicinity of an end portion opposite to the pulling direction and covers at least a front surface region of the organism contact surface, and a second paste layer attached to the front surface of the second base film, the second paste layer having an adhesive force which is peelable with respect to the skin by pulling the second base film toward the pulling direction.

4. The iontophoresis device according to claim 1, wherein the base film is provided on a front surface of the release liner integrally therewith, and is bent into a U shape at the vicinity of the end portion to be extended.

5. The iontophoresis device according to claim 4, further comprising:
a second release liner which is attached to a front surface of the paste layer so as to cover the paste layer, and is peelable by pulling the second release liner along the paste layer; and
a second base film which is provided on a front surface side of the second release liner, in the paste layer, which is connected to the second release liner at least at a vicinity of an end portion opposite to the pulling direction and covers at least a front surface region of the organism contact surface, and a second paste layer attached to the front surface of the second base film, the second paste layer having an adhesive force which is peelable with respect to the skin by pulling the second base film toward the pulling direction.

6. The iontophoresis device according to claim 1, further comprising:
a second release liner which is attached to a front surface of the paste layer so as to cover the paste layer, and is peelable by pulling the second release liner along the paste layer; and
a second base film which is provided on a front surface side of the second release liner, in the paste layer, which is connected to the second release liner at least at a vicinity of an end portion opposite to the pulling direction and covers at least a front surface region of the organism contact surface, and a second paste layer attached to the front surface of the second base film, the second paste layer having an adhesive force which is peelable with respect to the skin by pulling the second base film toward the pulling direction.

7. The iontophoresis device according to claim 6, wherein the second base film is formed by bending the second release liner into a U shape at the vicinity of the end portion and by extending the release liner.

8. The iontophoresis device according to claim 6, wherein the second base film is provided on a front surface of the second release liner integrally therewith, and is bent into a U shape at the vicinity of the end portion to be extended.

9. An iontophoresis device, comprising: a power source; and a working side electrode assembly and a non-working side electrode assembly connected to the power source, for administering a drug ion held in the working side electrode assembly to an organism from an organism contact surface at a tip of the working side electrode assembly through voltage applied from the power source, wherein:
the iontophoresis device further comprises:
a paste layer-attached release liner having a substantially same shape and size with the organism contact surface, one side end of the release liner being attached to one end portion of the organism contact surface; and
a winding member attached to another side end which is opposite to the one side end of the paste layer-attached release liner, capable of winding the paste layer-attached release liner from the other side end toward the one side end on one side, and
the paste layers are provided on a surface that is brought into contact with the winding member in the paste layer-attached release liner and a surface opposite thereto, the paste layers having an adhesive force which is peelable with respect to the skin when the paste layer-attached release liner is rolled up.

10. The iontophoresis device according to claim 9, wherein the organism contact surface comprises an organism contact surface at a tip of the non-working side electrode assembly.

11. An iontophoresis device, comprising:
a working side electrode assembly having an organism contact surface and a drug holding portion;
a non-working side electrode assembly having an organism contact surface, the working side electrode assembly and the non-working side electrode assembly operable to administer drug ions to an organism in response to a voltage applied across the working side and non-working side electrode assemblies;
a first selectively peelable member having a release liner portion and a base film portion, the release liner portion of the first selectively peelable member positioned such that a portion of the release liner portion covers the organism contact surface of at least the working side electrode prior to a peeling of the first selectively peelable member and which exposes at least the portion of the organism contact surface of at least the working side electrode after the peeling of the first selectively peelable member, and the base film portion of the first selectively peelable member disposed relatively outward of the release liner portion prior to the peeling of the first selectively peelable member to be disposed between the release liner portion and skin; and
a first adhesive layer carried on a portion of the base film portion of the first selectively peelable membrane, the first adhesive layer having an adhesive force that is peelable with respect to the skin while retaining at least some of an outer portion of the skin adhered thereto.

12. The iontophoresis device of claim 11 wherein a portion of the release member portion of the first selectively peelable member extends outward to form a pull tab.

13. The iontophoresis device according to claim 11, wherein the first selectively peelable member is U-shaped and the release liner portion and the base film portion are respective legs of the U-shaped first selectively peelable member.

14. The iontophoresis device according to claim 11 wherein the adhesive force of the first adhesive layer is such that the first adhesive layer remains adhered to the first peelable member after the peeling.

15. The iontophoresis device according to claim 11 wherein the first selectively peelable member comprises a portion of the base film portion carried on a portion of a front surface of the release liner portion.

16. The iontophoresis device according to claim 11, further comprising:
a second selectively peelable member having a release liner portion and a base film portion, the release liner portion of the second selectively peelable member attached to a front surface of the first adhesive layer such that at least a portion of the release liner portion of the second selectively peelable member covers the first adhesive layer prior to a peeling of the second selectively peelable member and which exposes the first adhesive layer after the peeling of the second selectively peelable member, the base film portion of the second selectively peelable member disposed relatively outward of the release liner portion of the second selectively peelable member prior to the peeling of the second selectively peelable member to be disposed between the release liner portion and skin; and
a second adhesive layer carried on a portion of the base film portion of the second selectively peelable membrane, the second adhesive layer having an adhesive force that is peelable with respect to the skin while retaining at least some of the outer portion of the skin adhered thereto.

17. The iontophoresis device of claim 16 wherein a portion of the release member portion of the second selectively peelable member extends outward to form a pull tab.

18. The iontophoresis device according to claim 16 wherein the second selectively peelable member is U-shaped and the release liner portion and the base film portion are respective legs of the U-shaped second selectively peelable member.

19. The iontophoresis device according to claim 16 wherein the second selectively peelable member comprises a portion of the base film portion carried on a portion of a front surface of the release liner portion.

20. The iontophoresis device according to claim 16 wherein the adhesive force of the second adhesive layer is such that the second adhesive layer remains adhered to the second peelable member after the peeling.

21. The iontophoresis device according to claim 11, further comprising:
a power source electrically coupled to the working side electrode assembly and the non-working side electrode assembly to provide a voltage thereacross.

22. An iontophoresis device, comprising:
a working side electrode assembly having an organism contact surface and a drug holding portion;
a non-working side electrode assembly having an organism contact surface, the working side electrode assembly and the non-working side electrode assembly operable to administer drug ions to an organism in response to a voltage applied across the working side and non-working side electrode assemblies;
an elongated release liner having a first end, a second end, and having a lateral dimension at least equal to a lateral dimension of the organism contact surfaces of the working and the non-working electrode assemblies, the release liner attached at least proximate the first end thereof at least proximate one portion of the organism contact surface of one of the working and non-working electrode assemblies;
at least one adhesive layer carried by at least a portion of a surface of the release liner, adhesive layer having an adhesive force that is peelable with respect to skin while retaining at least some of an outer portion of the skin adhered thereto and while the adhesive layer remains adhered to the portion of the surface of the release liner; and
a winding member, the release liner attached at least proximate the second end to a portion of the winding member, the winding member selectively operable to wind the attached release liner.

23. The iontophoresis device according to claim 22, further comprising:
a power source electrically coupled to the working side electrode assembly and the non-working side electrode assembly to provide a voltage thereacross.
